(19) United States
(12) Patent Application Publication
(10) Pub. No.: US 2026/0108736 A1
(43) Pub. Date: Apr. 23, 2026

(54) INTELLIGENT INTERACTIVE NEUROMODULATION SYSTEMS BY INTRAVESICAL AND PELVIC FLOOR STIMULATION

(71) Applicants: CHINA REHABILITATION SCIENCE INSTITUTE, Beijing (CN); CHINA REHABILITATION RESEARCH CENTER, Beijing (CN)

(72) Inventor: Limin LIAO, Beijing (CN)

(73) Assignees: CHINA REHABILITATION SCIENCE INSTITUTE, Beijing (CN); CHINA REHABILITATION RESEARCH CENTER, Beijing (CN)

(21) Appl. No.: 19/337,896

(22) Filed: Sep. 23, 2025

(30) Foreign Application Priority Data
Oct. 23, 2024 (CN) 202411480143.7

(51) Int. Cl. A61N 1/36; A61B 5/00; A61B 5/296; A61B 5/395; G16H 20/30; G16H 40/63; G16H 50/20

(52) U.S. Cl. CPC A61N 1/36031; A61B 5/296; A61B 5/395; A61B 5/7228; A61B 5/725; A61B 5/7257; A61B 5/7267; A61B 5/7289; A61N 1/36007; A61N 1/36017; G16H 20/30; G16H 40/63; G16H 50/20

(57) ABSTRACT

An intelligent interactive neuromodulation system by intravesical and pelvic floor stimulation is provided, including: an electromyography (EMG) acquisition module, an intelligent diagnostic module, a display interaction module, and an electrical stimulation module. The intelligent diagnostic module is configured to receive an original EMG signal acquired by the EMG acquisition module and obtain feature information, generate a first treatment protocol by using the feature information and a preconfigured intelligent fitting model, and generate a classification result of an abnormal bladder activity based on the feature information and a preconfigured intelligent classification model; in response to identifying that a detrusor activity of a patient changes, automatically complete dynamic switching between a first treatment mode and a second treatment mode. The display interaction module is configured to receive a first treatment protocol and a second treatment protocol, and send the first treatment protocol to the electrical stimulation module to apply electrical stimulation pulses.

### CROSS-REFERENCE TO RELATED APPLICATIONS

This application claims priority to the Chinese Patent Application No. 202411480143.7, filed on Oct. 23, 2024, the contents of which are hereby incorporated by reference.

### TECHNICAL FIELD

The present disclosure relates to a field of neuroelectric stimulation technology, and in particular to an intelligent interactive neuromodulation system by intravesical and pelvic floor stimulation.

### BACKGROUND

Intravesical electrical stimulation or intravesical neuromodulation is a treatment manner in which a stimulation catheter is inserted into the bladder, physiological saline is used as a medium to stimulate the detrusor muscle, and a remaining afferent neural pathway between the detrusor muscle and the central nervous system is used to induce the bladder to generate a urination sensation, so as to increase the transmission of nerve impulses, promote urination or enhance urinary control. At present, electrical stimulation therapy has been clinically confirmed to be an effective process for treating underactive bladder (UAB) symptoms. Meanwhile, clinical practice has found that, when combined with other relevant accessories, the process has a certain therapeutic effect on patients with overactive bladder (OAB) and patients with multiple system atrophy (MSA) presenting detrusor hyperactivity with impaired contractility (DHIC).

However, due to a plurality of different indications and changes in short-term urodynamics among individuals, existing electrical stimulation treatment protocols have difficulty achieving closed-loop neuromodulation and precise control of intravesical electrical stimulation. Therefore, there is a need to provide an intelligent interactive neuromodulation system by intravesical and pelvic floor stimulation, which can dynamically adjust a current protocol in real-time to better adapt to individual differences among different patients.

### SUMMARY

One or more embodiments of the present disclosure provide an intelligent interactive neuromodulation system by intravesical and pelvic floor stimulation by intravesical and pelvic floor stimulation, wherein the system comprises: an electromyography (EMG) acquisition module, an intelligent diagnostic module, a display interaction module, and an electrical stimulation module, the electrical stimulation module includes a first electrical stimulation unit and a second electrical stimulation unit, the EMG acquisition module is connected with the intelligent diagnostic module and the display interaction module, the electrical stimulation module is connected with the display interaction module, wherein the EMG acquisition module includes a first patch electrode attached to a perianal region, the first electrical stimulation unit includes an electrode inserted into the bladder lumen, the second electrical stimulation unit includes a second patch electrode attached to the perianal region. The intelligent diagnostic module is configured to: receive an original EMG signal acquired by the EMG acquisition module; obtain, based on the original EMG signal, feature information for quantifying a variation condition of the EMG signal during underactive bladder (UAB) and/or overactive bladder (OAB); generate a first treatment protocol by using the feature information and a preconfigured intelligent fitting model, and generate a classification result of an abnormal bladder activity based on the feature information and a preconfigured intelligent classification model; in response to identifying that a detrusor activity of a patient changes, automatically complete dynamic switching between a first treatment mode and a second treatment mode, wherein the first treatment mode includes: in response to determining that there is UAB, applying corresponding electrical stimulation pulses by using the electrode inserted into the bladder lumen based on the received first treatment protocol or a second treatment protocol input by physicians; the second treatment mode includes: in response to determining that there is OAB, applying corresponding electrical stimulation pulses by using the second patch electrode attached to the perianal region based on the received first treatment protocol or the second treatment protocol input by the physicians. The intelligent diagnostic module is further configured to: in response to determining that the patient presents with symptoms of the UAB, use the first treatment mode to perform electrical stimulation therapy using the electrode inserted into the bladder lumen; in response to determining that an EMG activity of the patient changes, determine that a current UAB is changed to the OAB, automatically switch and use the second treatment mode, deactivate the electrical stimulation pulses by using the electrode inserted into the bladder lumen, and activate the electrical stimulation pulses by using the second patch electrode attached to the perianal region; and in response to determining that the EMG activity of the patient changes, determine that a current OAB is changed to the UAB, automatically switch and use the first treatment mode, deactivate the second patch electrode attached to the perianal region, and activate the electrical stimulation pulses by using the electrode inserted into the bladder lumen.

### BRIEF DESCRIPTION OF THE DRAWINGS

The drawings described herein are intended to provide a further understanding of the present disclosure, and form a part of the present disclosure. The illustrative embodiments and descriptions of the present disclosure are used to explain the present disclosure, and do not constitute an improper limitation to the present disclosure. In the drawings:

FIG. 1 is a schematic diagram illustrating a structure of an intelligent interactive neuromodulation system by intravesical and pelvic floor stimulation according to some embodiments of the present disclosure;

FIG. 2 is a schematic diagram illustrating a first waveform of urodynamic measurement with synchronized electromyography (EMG) acquisition according to some embodiments of the present disclosure;

FIG. 3 is a schematic diagram illustrating a second waveform of urodynamic measurement with synchronized electromyography (EMG) acquisition according to some embodiments of the present disclosure;

FIG. 4 is a schematic diagram illustrating a third waveform of urodynamic measurement with synchronized electromyography (EMG) acquisition according to some embodiments of the present disclosure;

FIG. 5 is an exemplary flowchart illustrating generating a first treatment protocol according to some embodiments of the present disclosure;

FIG. 6 is an exemplary schematic diagram illustrating a feature extraction model according to some embodiments of the present disclosure;

In the figure: 1. EMG Acquisition Module; 2. Intelligent Diagnostic Module; 3. Display Interaction Module; 4. Electrical Stimulation Module; 41. First Electrical Stimulation Unit; 42. Second Electrical Stimulation Unit; 5. Server Module; 21. Intelligent Classification Model Unit; 22. Intelligent Fitting Model Unit; 31. Original Signal Unit; 32. EMG Signal Unit; 33. Feature Information Unit; 34. Algorithm Configuration Unit; 35. Treatment Protocol Configuration Unit.

### DETAILED DESCRIPTION

In the following detailed description, numerous specific details are set forth by way of examples in order to provide a thorough understanding of the relevant disclosure. Obviously, drawings described below are only some examples or embodiments of the present disclosure. Those skilled in the art, without further creative efforts, may apply the present disclosure to other similar scenarios according to these drawings.

To facilitate the understanding of the technical solutions of an intelligent interactive neuromodulation system by intravesical and pelvic floor stimulation in the embodiments of the present disclosure, the technical principles of the embodiments of the present disclosure are first described.

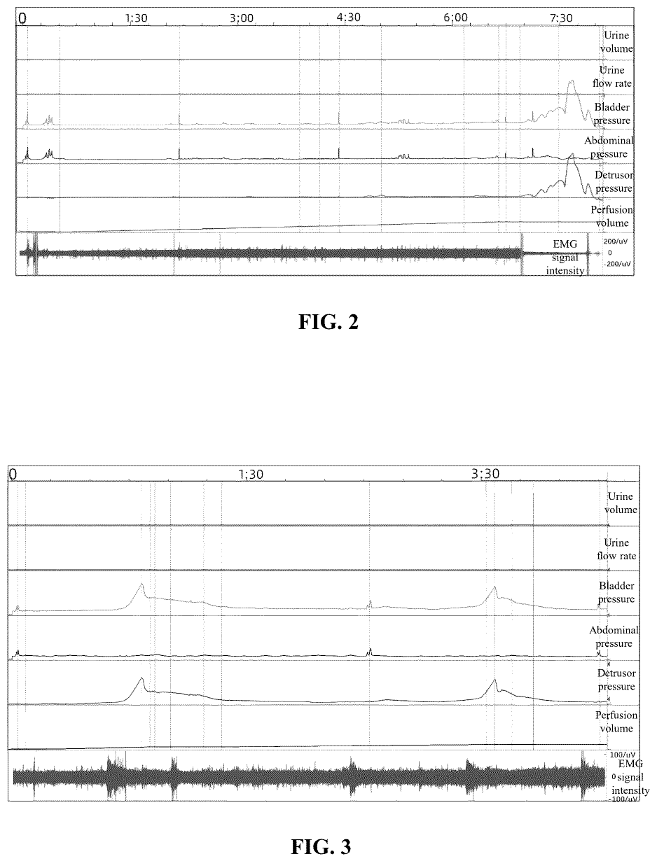

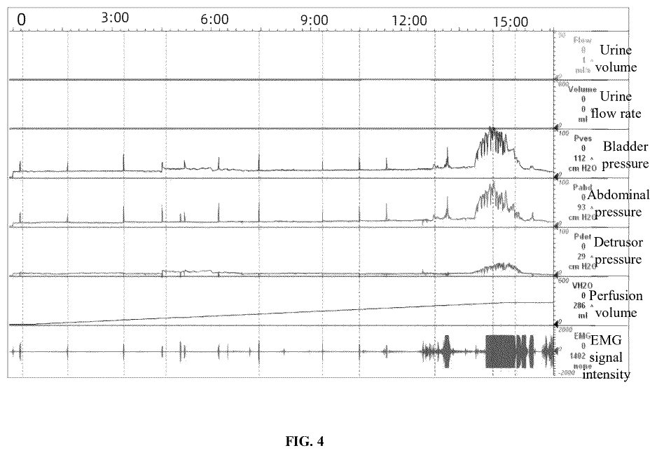

FIG. 2 is a schematic diagram illustrating a first waveform of urodynamic measurement with synchronized electromyography (EMG) acquisition according to some embodiments of the present disclosure. FIG. 3 is a schematic diagram illustrating a second waveform of urodynamic measurement with synchronized electromyography (EMG) acquisition according to some embodiments of the present disclosure. FIG. 4 is a schematic diagram illustrating a third waveform of urodynamic measurement with synchronized electromyography (EMG) acquisition according to some embodiments of the present disclosure.

In conjunction with FIG. 2 to FIG. 4, the present disclosure provides schematic diagrams of waveforms for urodynamic measurements under different conditions. In FIG. 2 to FIG. 4, the horizontal axis represents time (min), and the vertical axis sequentially represents urine volume (ml), urine flow rate (ml/s), bladder pressure (cmH₂O), abdominal pressure (cmH₂O), detrusor pressure (cmH₂O), perfusion volume (ml), and EMG signal intensity (μV) from top to bottom.

As shown in FIG. 2, under normal conditions, during a bladder filling phase (also referred to as a storage phase), as bladder volume increases, the bladder pressure shows no significant increase or only a small increase. Meanwhile, pelvic floor electromyographic signals (also referred to as EMG signals) recorded from the perianal region remain stable at a certain amplitude without obvious changes. During a micturition phase (also referred to as a voiding phase or a bladder-empty phase), the detrusor muscle undergoes voluntary contraction, and the pelvic floor EMG signals recorded from the perianal region significantly weaken to allow smooth urine expulsion. As shown in FIG. 3 and FIG. 4, when a patient has lower urinary tract dysfunction, the EMG signals may be divided into the following two situations: Situation 1, during the bladder filling phase, the patient experiences detrusor uninhibited contraction, and the pelvic floor EMG signals recorded from the perianal region show a simultaneous increase, manifesting as detrusor hyperactivity during the bladder filling phase, which may be specifically referred to in FIG. 3. Situation 2, during the micturition phase, when the patient has voluntary detrusor contraction, the pelvic floor EMG signals recorded from the perianal region show a simultaneous increase, leading to impaired urine expulsion and manifesting as difficulty in urination, which may be specifically referred to in FIG. 4.

Therefore, the technical concept of the present disclosure is that the intelligent interactive neuromodulation system by intravesical and pelvic floor stimulation, based on electromyographic feedback technology, utilizes the correlation between pelvic floor EMG signals and detrusor activity to collect and recognize an intensity variation condition of the pelvic floor EMG signals from the patient and clinical feature information of the patient in real time. In response to identifying that the detrusor activity of the patient changes, the system automatically completes dynamic switching between a first treatment mode and a second treatment mode. That is, the system automatically completes the dynamic switching between different treatment modes according to a variation condition of the EMG signal during underactive bladder (UAB) and overactive bladder (OAB). By establishing an intelligent diagnostic module, the system realizes automatic and semi-automatic adjustment functions of a neuromodulation system, so as to output a first treatment protocol automatically generated or a second treatment protocol input by physicians to an electrical stimulation module. Thereby, the system realizes closed-loop neuromodulation and precise control of EMG signal acquisition and intravesical electrical stimulation, achieving technical effects of improving the accuracy of treatment protocols, enhancing the intelligence and flexibility of the system, and reducing the burden on physicians.

The technical solutions provided in the embodiments of the present disclosure are described in detail below in conjunction with the drawings.

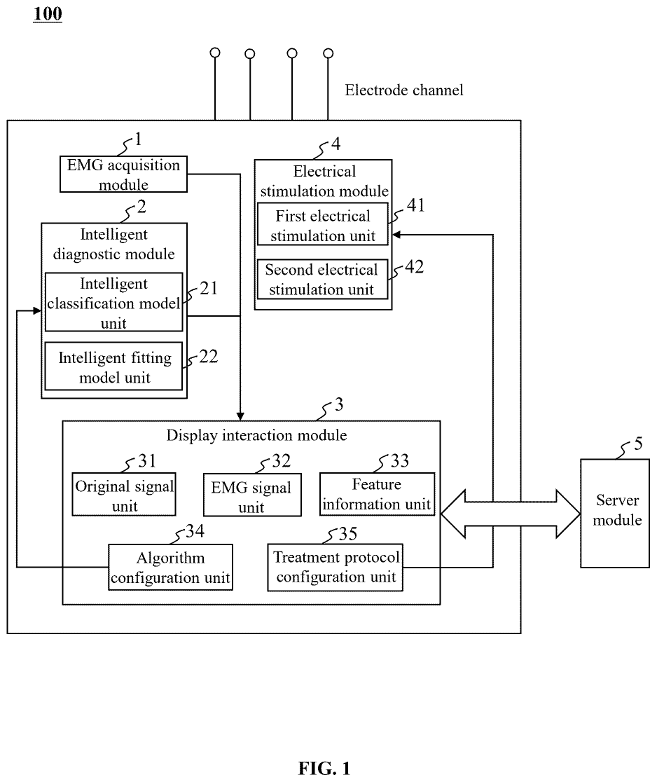

FIG. 1 is a schematic diagram illustrating a structure of an intelligent interactive neuromodulation system by intravesical and pelvic floor stimulation according to some embodiments of the present disclosure.

Embodiments of the present disclosure provide an intelligent interactive neuromodulation system by intravesical and pelvic floor stimulation. As shown in FIG. 1, a schematic diagram of a structure of the intelligent interactive neuromodulation system 100 by intravesical and pelvic floor stimulation is provided. The intelligent interactive neuromodulation system 100 by intravesical and pelvic floor stimulation of the embodiment of the present disclosure includes: an EMG acquisition module 1, an intelligent diagnostic module 2, a display interaction module 3, and an electrical stimulation module 4. The electrical stimulation module 4 includes a first electrical stimulation unit 41 and a second electrical stimulation unit 42. The EMG acquisition module 1 is connected with the intelligent diagnostic module 2 and the display interaction module 3, and the electrical stimulation module 4 is connected with the display interaction module 3. The EMG acquisition module 1 includes a first patch electrode attached to a perianal region, the first electrical stimulation unit includes an electrode inserted into the bladder lumen, the second electrical stimulation unit includes a second patch electrode attached to the perianal region.

In some embodiments, one or more of the EMG acquisition module 1, the intelligent diagnostic module 2, the display interaction module 3, and the electrical stimulation module 4 may be integrated into a processor. The processor may be used to manage data resources, and process data and/or information from at least one component involved in the intelligent interactive neuromodulation system by intravesical and pelvic floor stimulation or external data sources. The processor may execute program instructions based on such data, information, and/or processing results, thereby performing one or more of the functions described in the present disclosure. In some embodiments, the processor may include one or more sub-processing devices (e.g., a single-core processing device or a multi-core processing device). Merely by way of example, the processor may include a central processing unit (CPU), an application-specific integrated circuit (ASIC), a microprocessor, etc., or any combination of the above.

In some embodiments, the first patch electrode refers to an electrode directly attached to the perianal region, which is used for collecting EMG signals. For example, the first patch electrode includes an electrode configured with signal acquisition functions and a stimulation electrode, or the like. The second patch electrode refers to an electrode directly attached to the perianal region, which is used for stimulating the perianal region to achieve neuromodulation of pelvic function of the patient. An insertable stimulation electrode is further disposed within the bladder for modulating the function of the patient's bladder through electrical stimulation. In some embodiments, the electrical stimulation module 4 may also include stimulation electrodes arranged at other positions to apply electrical stimulation pulses to different positions for different symptoms of the patient, respectively, or the like. A count of the first patch electrodes, the second patch electrodes, and the electrodes inserted into the bladder lumen may be preset according to actual needs.

In some embodiments, the EMG acquisition module 1 is configured to acquire original EMG signals from the perianal region, vagina, abdominal muscles, and other parts of the patient via the electrodes such as the first patch electrode. The original EMG signal refers to an EMG signal directly obtained from muscles without any processing. The EMG acquisition module 1 is configured as a signal acquisition device such as an EMG signal acquisition instrument.

In some embodiments, the intelligent diagnostic module 2 is configured to: receive the original EMG signal acquired by the EMG acquisition module 1; obtain, based on the original EMG signal, feature information for quantifying a variation condition of the EMG signal during underactive bladder (UAB) and/or overactive bladder (OAB); generate a first treatment protocol by using the feature information and a preconfigured intelligent fitting model, and generate a classification result of an abnormal bladder activity based on the feature information and a preconfigured intelligent classification model. The intelligent diagnostic module 2 is configured as a digital signal processor.

The feature information refers to information related to the detrusor activity of the patient characterized by the original EMG signals. For example, the feature information includes information such as a root mean square (RMS) value, a contraction or relaxation time of a muscle, and an intensity variation of the EMG signal. The feature information may quantify the variation condition of the EMG signal during UAB and/or OAB. In some embodiments, the intelligent diagnostic module 2 may receive the original EMG signals and extract the feature information therefrom.

In some embodiments, the intelligent fitting model may be a machine learning model, such as a neural network model, a Bayesian polynomial fitting model, or any other custom model structure, or a combination thereof. In some embodiments, inputs to the intelligent fitting model may include the feature information, and outputs may include the first treatment protocol. The intelligent fitting model may be obtained through machine learning training by a server module.

More descriptions regarding the intelligent fitting model and the server module may be found in the related descriptions below.

The first treatment protocol refers to a protocol automatically generated by the intelligent fitting model. The first treatment protocol includes information such as treatment parameters and treatment time. The treatment parameters include data such as frequency, amplitude, pulse width, or the like of stimulating muscles using the electrode.

The intelligent classification model may be a machine learning model, such as a decision tree, a random forest, or any other custom model structure, or a combination thereof. In some embodiments, inputs to the intelligent classification model may include the feature information, and outputs may include the classification results of normal detrusor activity or abnormal bladder activity. The intelligent fitting model may be obtained through machine learning training by the server module.

The classification results of the abnormal bladder activity may include classification results of various urinary tract dysfunction symptoms such as OAB symptoms, UAB symptoms, or detrusor hyperactivity with impaired contractility (DHIC) symptoms.

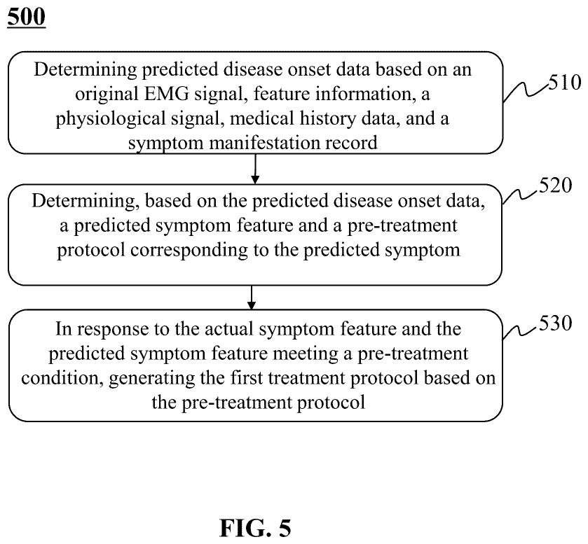

In some embodiments, the intelligent diagnostic module 2 is further configured to determine the first treatment protocol based on the original EMG signals, the feature information, physiological signals, medical history data, the disease manifestation records, or the like. More descriptions regarding the determination of the first treatment protocol may be found in FIG. 5 and relevant descriptions thereof.

In some embodiments, in response to identifying that a detrusor activity of a patient changes, the intelligent diagnostic module 2 automatically completes dynamic switching between a first treatment mode and a second treatment mode, wherein the first treatment mode includes: in response to determining that there is UAB, applying corresponding electrical stimulation pulses by using the electrode inserted into the bladder lumen based on the received first treatment protocol or a second treatment protocol input by physicians; the second treatment mode includes: in response to determining that there is OAB, applying corresponding electrical stimulation pulses by using the second patch electrode attached to the perianal region based on the received first treatment protocol or the second treatment protocol input by the physicians.

In some embodiments, the intelligent diagnostic module 2 may apply the corresponding electrical stimulation pulses by using the electrode inserted into the bladder lumen based on the received first treatment protocol generated by the intelligent fitting model or the second treatment protocol input by the physicians through the display interaction module.

In some embodiments, changes in the detrusor activity of the patient may include at least one of a change in the detrusor activity of the patient from normal activity to abnormal activity, a change in the type of abnormal bladder activity, or the like. For example, the change in the detrusor activity of the patient may include a change from the UAB symptom to the OAB symptom, a change from the OAB symptom to the UAB symptom, or the like. The intelligent diagnostic module 2 may obtain the output of the intelligent fitting model in real-time to determine whether there is a change in the detrusor activity of the patient. In response to identifying that the detrusor activity of the patient changes, the intelligent diagnostic module 2 may switch treatment modes and administer treatment to the patient according to either the first treatment mode or the second treatment mode.

The embodiments of the present disclosure may realize the dynamic switching between the two treatment modes according to the variation condition in the detrusor activity of the patient.

In some embodiments, in response to determining that the patient presents with symptoms of UAB, the intelligent diagnostic module 2 uses the first treatment mode, i.e., performing electrical stimulation therapy using the electrode inserted into the bladder lumen of the patient. In response to determining that an EMG activity of the patient changes, the intelligent diagnostic module 2 determines that a current UAB is changed to OAB, automatically switches and uses the second treatment mode, deactivates the electrical stimulation pulses using the electrode inserted into the bladder lumen, and activates the electrical stimulation pulses using the second patch electrode attached to the perianal region. In response to determining that the EMG activity of the patient changes, the intelligent diagnostic module 2 determines that a current OAB is changed to UAB, automatically switches and uses the first treatment mode, closes the second patch electrode attached to the perianal region, and activates the electrical stimulation pulses by using the electrode inserted into the bladder lumen.

As described above, by analogy, the dynamic switching between the two treatment modes is realized.

In some embodiments, in response to the output of the intelligent classification model being a UAB symptom, the intelligent diagnostic module 2 may determine that the patient presents with symptoms of UAB.

In some embodiments, changes in the EMG activity of the patient may include at least one of an enhancement of the EMG signal, a change in the feature information, or the like. Another change in the EMG activity of the patient may include a return of the EMG signal to the original low signal state, etc.

In some embodiments of the present disclosure, the above approach enables the dynamic switching between the first treatment mode and the second treatment protocol for UAB symptoms and OAB symptoms, thereby improving the level of intelligent and precise treatment.

The embodiments of the present disclosure provide the intelligent interactive neuromodulation system by intravesical and pelvic floor stimulation, aiming to autonomously evaluate the current clinical feature information of the patient, determine abnormal bladder symptoms, automatically achieve the dynamic switching between different treatment modes, promptly implement corresponding treatment plans, improve the precision of treatment protocols, and reduce the burden on physicians.

Different from technical solutions that utilize ultrasound technology to monitor bladder pressure and volume for electrical stimulation pulses but cannot achieve the dynamic switching, the intelligent interactive neuromodulation system by intravesical and pelvic floor stimulation in the embodiments of the present disclosure can collect the original EMG signals of the patient in real-time, process and recognize the original EMG signals, and autonomously evaluate and monitor the current clinical feature information of the patient. When changes in the detrusor activity of the patient are detected, the system adaptively and dynamically switches between treatment modes for UAB symptoms and OAB symptoms, thereby promptly implementing targeted treatment protocols. Moreover, the system eliminates the need for physicians to monitor a state of the patient in real-time, achieving precise treatment. The system can not only achieve closed-loop neuromodulation and precise control between the EMG signal acquisition and electrical stimulation modules, effectively improving treatment efficiency and effect, but also make treatment for different patients more intelligent and personalized, which greatly reduces the burden on the physicians.

In some embodiments, the intelligent diagnostic module may dynamically switch the first treatment protocol based on the EMG signals and feature information collected during a treatment process. Dynamically switching the first treatment protocol based on the EMG signals and feature information collected during the treatment process includes: in response to the EMG signals from the perianal region of the patient not meeting a preset change requirement within a first preset time period, increasing basic parameters of the first treatment protocol.

The first preset time period refers to a preset duration used for a single treatment process.

In some embodiments, the processor may use an average duration of a single historical treatment process across a plurality of historical treatment processes for a corresponding symptom as the first preset time period.

The corresponding symptom refers to a symptom targeted by the treatment process, such as overactive bladder (OAB) symptoms, underactive bladder (UAB) symptoms, detrusor hyperactivity with impaired contractility (DHIC) symptoms, or the like.

In some embodiments, the first preset time period may be set by a technician based on experiences.

The preset change requirement refers to an amplitude variation of the EMG signals within the first preset time period meeting a preset amplitude threshold. For example, the amplitude variation is greater than the preset amplitude threshold.

The amplitude variation refers to a range of changes in the intensity of the EMG signals. In some embodiments, the processor may determine the amplitude variation by analyzing the EMG signal intensity within the first preset time period. In some embodiments, the processor may determine the amplitude variation based on changes in the EMG signal intensity from a waveform diagram obtained via urodynamic measurement. More descriptions regarding the waveform diagram of the urodynamic measurement may be found in FIGS. 2-4 and relevant descriptions thereof.

The preset amplitude refers to a variation amplitude of EMG signals that achieves the expected therapeutic effect.

In some embodiments, the preset change requirement may be set by a technician based on experience.

In some embodiments, the first preset time period and the preset change requirement are related to the severity of the patient's symptoms.

The severity of the patient's symptoms may be characterized by a frequency of symptom occurrence in symptom records of the patient.

In some embodiments, the symptom records of the patient may be obtained from medical records.

In some embodiments, the intelligent diagnostic module may determine the severity of the patient's symptoms based on the frequency of symptom occurrence in the symptom records of the patient. For example, the higher the frequency of occurrence of the patient's symptoms, the more severe the patient's symptoms. By way of example, the intelligent diagnostic module may normalize the frequency of occurrence of the patient's symptoms, which is mapped to a value ranging from 0 to 1 to represent the severity of the patient's symptoms. The higher the value, the more severe the patient's symptoms.

In some embodiments, the intelligent diagnostic module may adjust the first preset time period according to the severity of the patient's symptoms. For example, the more severe the patient's symptoms are, the longer the first preset time period is; conversely, the less severe the patient's symptoms are, the shorter the first preset time period is.

In some embodiments, the intelligent diagnostic module may adjust the preset change requirement based on the severity of the patient's symptoms. For example, if the severity of the patient's symptoms is relatively high, the preset amplitude in the preset change requirement may be appropriately reduced; conversely, if the severity of the patient's symptoms is relatively low, the preset amplitude in the preset change requirement may be appropriately increased.

Relating the first preset time period and the preset change requirements to the severity of the patient's symptoms enables the adjustment strategy of the first treatment protocol to be personalized and adapted to the severity of the patient's symptoms, which makes the first treatment protocol more suitable for the patient's specific conditions, achieving the effect of ensuring that patients with severe symptoms receive sufficient treatment intensity while preventing over-treatment of patients with mild symptoms.

In some embodiments, the basic parameters of the first treatment protocol include the amplitude, pulse width, frequency, treatment duration, and voltage/current source of electrical stimulation pulses. More descriptions regarding the basic parameters may be found in the related descriptions below.

In some embodiments, during the treatment process, the intelligent diagnostic module may collect the EMG signals in real-time and determine the feature information. The intelligent diagnostic module may adjust the basic parameters of the first treatment protocol in real-time based on the amplitude variation of the EMG signals and the adjustment range of the basic parameters. The adjustment range refers to an adjustment difference value for each item in the basic parameters. For example, in response to the amplitude variation of the EMG signals of the patient being less than or equal to the preset amplitude, the intelligent diagnostic module increases the basic parameters of the first treatment protocol according to the corresponding adjustment range. For another example, in response to the amplitude variation of the EMG signals from the perianal region of the patient being greater than the preset amplitude, i.e., the variation of the EMG signals from the perianal region of the patient within the first preset time period meeting the preset change requirement, the intelligent diagnostic module may maintain the basic parameters of the first treatment protocol, or the like.

In some embodiments, the adjustment range may be positively correlated with the severity of the patient's symptoms. For example, the higher the severity of the patient's symptoms, the greater the adjustment range.

In some embodiments, the adjustment range may be positively correlated with the basic parameters. For example, the intelligent diagnostic module may set the adjustment range according to a percentage of the basic parameters. In some embodiments, the specific value of the percentage may be set by a technician based on experience.

In some embodiments, the intelligent diagnostic module may obtain the adjustment range of the basic parameters based on a preset table. The preset table reflects corresponding relationships among the amplitude variation of EMG signals, the feature information, the basic parameters, and the adjustment range. In response to the EMG signals from the perianal region of the patient not meeting the preset change requirement within the first preset time period, the intelligent diagnostic module may determine the adjustment range of the basic parameters by querying the preset table based on the amplitude variation of the EMG signals, the feature information, and the basic parameters.

In some embodiments, the preset table may be set by a technician based on experience.

The intelligent diagnostic module dynamically adjusts the first treatment protocol based on the EMG signals and the feature information during the treatment process, which can respond to the actual reaction of the patient to the treatment in time, avoiding insufficient efficacy due to a fixed first treatment protocol. By automatically increasing the basic parameters of the first treatment protocol with poor efficacy, it can enhance the targeting of treatment, ensure that the treatment intensity matches the patient condition, and improve the efficiency of rehabilitation.

In some embodiments, the display interaction module 3 is configured to: receive the first treatment protocol, send the first treatment protocol to the electrical stimulation module (the first electrical stimulation unit 41 or the second electrical stimulation unit 42) to apply the corresponding electrical stimulation pulses; or, the display interaction module 3 is further configured to: receive the second treatment protocol input by the physicians based on the original EMG signal, the feature information, and the classification result received by the display interaction module, and send the second treatment protocol to the electrical stimulation module (the first electrical stimulation unit 41 or the second electrical stimulation unit 42) to apply the corresponding electrical stimulation pulses.

In some embodiments, the display interaction module 3 may be configured as a user terminal. The user terminal refers to one or more terminal devices or software used by a user. In some embodiments, the user terminal may be one or any combination of mobile devices, tablet computers, laptop computers, and other devices with input and/or output functions. In some embodiments, one or more users may use the user terminal. The users may include physicians, or the like.

The treatment protocols in the embodiments of the present disclosure may be specifically divided into two types. The first treatment protocol, namely a fully automatic adjustment protocol, refers to a treatment protocol that is automatically generated by the intelligent diagnostic module 2 using the feature information and the preconfigured intelligent fitting model. The intelligent diagnostic module 2 may send the first treatment protocol to the display interaction module 3. The treatment protocol configuration unit 35 of the display interaction module 3 sends the first treatment protocol to the first electrical stimulation unit 41 or the second electrical stimulation unit 42 to apply the corresponding electrical stimulation pulses. More descriptions regarding the treatment protocol configuration unit 35 may be found in the related descriptions below.

In some embodiments of the present disclosure, the display interaction module receives the first treatment protocol and sends the first treatment protocol to the first electrical stimulation unit or the second electrical stimulation unit to apply the corresponding electrical stimulation pulses, which eliminates the need for frequent manual adjustments, effectively improving the efficiency and effectiveness of the treatment.

In some embodiments, the second treatment protocol, i.e., a semi-automatic adjustment protocol, refers to a treatment protocol generated jointly by the classification results related to abnormal bladder activity and manual input by the physicians. In some embodiments, the intelligent diagnostic module 2 generates the classification results related to the abnormal bladder activity of the patient, such as classification results for various urinary tract dysfunction symptoms including the OAB symptoms, the UAB symptoms, or the DHIC symptoms. The intelligent diagnostic module 2 sends the original EMG signals, feature information, and classification results to the display interaction module 3. The physician makes a comprehensive judgment based on the information displayed by the display interaction module 3 and provides the second treatment protocol. Subsequently, the treatment protocol configuration unit 35 of the display interaction module 3 sends the second treatment protocol to the first electrical stimulation unit 41 or the second electrical stimulation unit 42 to apply the corresponding electrical stimulation pulses. In other words, in response to determining that the patient presents with symptoms of UAB, the intelligent diagnostic module 2 adopts the first treatment mode, sends the second treatment protocol to the first electrical stimulation unit 41, and applies the corresponding electrical stimulation pulses using the electrode inserted into the bladder lumen; in response to determining that the patient presents with symptoms of the OAB, the intelligent diagnostic module 2 adopts the second treatment mode, sends the second treatment protocol to the second electrical stimulation unit 42, and applies the corresponding electrical stimulation pulses using the second patch electrode attached to the perianal region.

In some embodiments of the present disclosure, the display interaction module 3 displays interactive interface information and receives the second treatment protocol input by the physician, so as to send the second treatment protocol to the first electrical stimulation unit or the second electrical stimulation unit and apply the corresponding electrical stimulation pulses, which enables the physician to make a comprehensive judgment through the display interaction module and provide a customized second treatment protocol, thereby improving the effectiveness and accuracy of the treatment.

It can be understood that the difference between the two treatment protocols lies in that treatment parameters of the first treatment protocol are automatically generated by the system, while treatment parameters of the second treatment protocol are manually input by the physician. Meanwhile, both the first treatment protocol and the second treatment protocol include treatment parameters for symptoms related to both the first treatment mode and the second treatment mode. Therefore, when generating the first treatment protocol, there is no need for physician participation or decision-making. The intelligent interactive neuromodulation system by intravesical and pelvic floor stimulation may automatically monitor and analyze the EMG signals of the patient, complete dynamic switching between treatment modes, and automatically output treatment parameters to ensure that the system outputs the optimal first treatment protocol. While the second treatment protocol relies to a certain extent on the intervention and decision-making of the physician. The intelligent interactive neuromodulation system by intravesical and pelvic floor stimulation may automatically monitor and analyze the EMG signals of the patient, automatically output a diagnostic conclusion, and complete dynamic switching between treatment modes. However, when outputting treatment parameters, it still requires the physician to make a comprehensive judgment based on the displayed information. Of course, no matter which treatment protocol is used, the core goal is to improve the intelligence and personalization of the entire system, thereby reducing the burden on medical staff and enhancing the treatment capacity and effectiveness.

In some embodiments of the present disclosure, two types of treatment protocols can be output respectively by adopting an automatic adjustment mechanism and a semi-automatic adjustment mechanism. Thus, a more appropriate treatment protocol can be selected for treatment according to the dynamic switching between treatment modes, which makes the treatment for different patients more intelligent, precise, and personalized, further improving the treatment effect.

Of course, the above descriptions regarding the two treatment modes and the two treatment protocols are merely exemplary embodiments and should not be construed as limitations to the present disclosure. Those skilled in the art may also combine the above two treatment protocols or improve them according to specific usage scenarios. For example, the treatment parameters input by the physician can be pre-stored in the intelligent classification model to directly output the second treatment protocol when used offline or when the physician is not present, that is, directly output the pre-stored standardized treatment protocol. For another example, after the intelligent fitting model automatically outputs the first treatment protocol, the physician can fine-tune the first treatment protocol, which will not be repeated here.

It should be noted that the technical solutions of the present disclosure are particularly suitable for the monitoring, symptom identification, evaluation, and treatment of patients with UAB symptoms, patients with OAB symptoms, and patients with detrusor hyperactivity and impaired contractility. It can be understood that patients with detrusor hyperactivity and impaired contractility refer to those who have both the OAB symptom and the UAB symptom. Main clinical features of patients with detrusor hyperactivity and impaired contractility include urinary frequency, urgency, and urge incontinence caused by detrusor overactivity during the bladder filling phase, while detrusor underactivity during the micturition phase leads to increased residual urine volume. Of course, the technical solutions of the present disclosure can also be extended to the monitoring and treatment of other urinary tract dysfunction symptoms, which are not limited here.

In some embodiments, the original EMG signal includes at least one of a vaginal surface EMG signal, an anal sphincter and perianal region EMG signal, and an abdominal surface EMG signal of a patient.

In some embodiments, an EMG acquisition unit is pre-arranged at positions such as the vagina, anal sphincter, perianal region, and surface of abdominal muscles of the patient to acquire signals.

The EMG acquisition unit refers to an acquisition unit in the EMG acquisition module for acquiring the EMG signals from each of the parts. For example, the EMG acquisition unit includes patch electrodes for acquiring the EMG signals, etc.

In some embodiments, the original EMG signals are divided into voluntary contraction EMG signals and involuntary contraction EMG signals. That is, the original EMG signals include the EMG signals acquired during the voluntary contraction phase of the pelvic floor muscles and the EMG signals acquired during the involuntary contraction phase of the pelvic floor muscles. The voluntary contraction EMG signal refers to an EMG signal feature acquired from the aforementioned different parts of the patient when the patient contracts the pelvic floor muscles under the guidance and instructions of the physician during a contraction process. The involuntary contraction EMG signal refers to an EMG signal feature of the pelvic floor muscles that is acquired at the aforementioned different parts of the patient during the micturition phase. In some embodiments, the voluntary contraction EMG signals are further divided into the EMG signals during four different periods: resting phase, fast muscle contraction, slow muscle sustained contraction (e.g., 5-10 seconds), and slow muscle endurance contraction (e.g., 60 seconds). By acquiring the original EMG signals during the above plurality of periods, the accuracy and precision of signal recognition and judgment of the intelligent interactive neuromodulation system by intravesical and pelvic floor stimulation are further improved.

In some embodiments, the intelligent diagnostic module 2 is further configured to: perform filter processing and Fourier transform processing on the original EMG signal to obtain the feature information for quantifying the variation condition of the EMG signal during UAB and/or OAB; wherein the filter processing is used to extract an EMG signal in a frequency range of 20 Hz-500 Hz from the original EMG signal, and signal instantaneous root mean square (RMS) values are calculated according to a preset sampling rate to perform window smoothing processing, a RMS variation curve after window smoothing processing is used to extract the feature information.

In some embodiments, the feature information of the variation condition of the EMG signal may also be referred to as the feature information of the original EMG signal.

The RMS variation curve may reflect changes in the EMG signal intensity over time. The filter processing refers to performing frequency screening and noise removal on the original EMG signals. The Fourier transform processing refers to a manner of performing frequency domain analysis on original EMG information. The Fourier transform processing may convert the filtered original EMG signal from time domain signals to frequency domain signals, obtain sinusoidal wave components of different frequencies, and generate a frequency domain graph. The frequency domain graph may reflect a frequency feature of EMG signals. The intelligent diagnostic module may analyze the intensity distribution of each frequency component based on the frequency domain graph.

The preset sampling rate may be set by a technician based on experience.

In some embodiments, the filter processing may also extract and process the EMG signals in each frequency range from the original EMG signal. The frequency range may be preset according to actual needs.

In some embodiments, the intelligent diagnostic module may obtain the feature information of the EMG signals in different periods by combining the RMS variation curve after window smoothing processing, the frequency domain graph, or the like.

In some embodiments, the feature information of the voluntary contraction EMG signal includes an average value of the EMG signals during the resting phase; a maximum value, contraction time, average rate of change, relaxation time, and average rate of change of the EMG signals during fast muscle contraction; an average value and normalized mean square error of the EMG signals during slow muscle sustained contraction; and an overall average value, normalized mean square error, and an average value in the last 20 seconds of the EMG signals during slow muscle endurance contraction, etc.

The last 20 seconds refer to final 20 seconds of the slow muscle endurance contraction phase. The average rate of change refers to an average slope from the start point of contraction to the peak signal of contraction. The feature information of involuntary contraction EMG signals includes but is not limited to ramp-up phase, a maximum value, ramp-down phase of the EMG signals, an average value during the resting phase, an average value of the EMG signals on the abdominal muscle surface, and a rate of change of the EMG signals, etc.

It can be known from the above that the feature information of the original EMG signal and the variation condition of the EMG signal in the embodiments of the present disclosure can, to a certain extent, characterize an abnormal bladder activity. In other words, by processing the original EMG signals, the feature information for quantifying the variation condition of the EMG signal during UAB and/or OAB is obtained, thereby enabling accurate identification and autonomous assessment of the patients'symptoms. For example, when collecting the perianal region EMG signals from the patient, if the EMG signals collected during the bladder filling phase show a sustained increase for a certain period of time, it indicates that detrusor hyperactivity has occurred; on the contrary, if the EMG signals collected during the micturition phase do not show a significant decrease compared with those before urination, it indicates detrusor underactivity or reduced detrusor contractility.

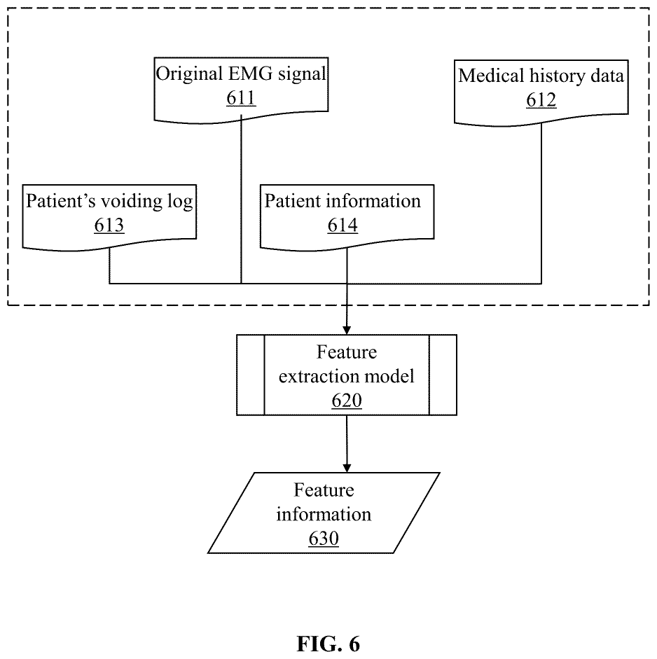

In some embodiments, the processor may obtain the feature information through a feature extraction model based on the original EMG signal, medical history data, the patient's voiding log, and the patient information, etc. More descriptions may be found in FIG. 6 and relevant descriptions thereof.

Of course, the above content related to the feature information of the original EMG signal and the variation condition of the EMG signal is only for the purpose of easy understanding and simplified description, and is not to be regarded as a limitation of the present disclosure.

In some embodiments, the intelligent interactive neuromodulation system by intravesical and pelvic floor stimulation further includes a server module 5. The server module is connected with the display interaction module 3. The server module is configured to: construct the intelligent fitting model and/or the intelligent classification model, and send a trained intelligent fitting model and/or intelligent classification model to the display interaction module 3. The intelligent fitting model and/or the intelligent classification model is trained by a training sample set via a machine learning algorithm. Training data in the training sample set includes clinical acquisition data, including an original EMG signal sent by the display interaction module 3, the feature information of the original EMG signal processed by a preset processing algorithm, etc.

The clinical acquisition data refers to data acquired that is related to clinical activities.

In some embodiments, the server module is configured as a server. The server may be configured to manage resources and process data and/or information from at least one component of the system or external data sources. In some embodiments, the server may be a single server or a server group. In some embodiments, the training sample set used for training the intelligent fitting model may include a sample set composed of a plurality of first training samples.

In some embodiments, the server module 5 may train the intelligent fitting model through a large number of first training samples and first labels corresponding to the first training samples. In some embodiments, the server module 5 may input a plurality of first training samples with first labels into the initial intelligent fitting model, construct a loss function based on the first labels and the results of the initial intelligent fitting model, and iteratively update the parameters of the initial intelligent fitting model through gradient descent or other methods based on the loss function. The model training is completed when a preset condition is satisfied, and the trained intelligent fitting model is obtained. The preset conditions may include convergence of the loss function, a count of iterations reaching a threshold, etc.

In some embodiments, the first training sample may include sample feature information. The first label is a sample first treatment protocol corresponding to the first training sample. In some embodiments, the first training sample and the first label may be determined based on the clinical acquisition data in historical data. The historical data may include historical feature information of a normal person and a patient. The historical feature information is obtained from historical original EMG signals through the preset processing algorithm. The first label may also be determined manually. For example, the first labels include an optimal treatment protocol determined by a physician, etc.

In some embodiments, the training sample set for training the intelligent classification model may include a sample set composed of a plurality of second training samples.

In some embodiments, the server module 5 may obtain the intelligent classification model through a large number of second training samples and second labels corresponding to the second training samples. The intelligent classification model is trained in a manner similar to that of the intelligent fitting model, which will not be repeated here.

In some embodiments, the second training sample may include sample feature information. The second label is a sample classification result corresponding to the second training sample. In some embodiments, the second training sample is acquired in a similar manner as the first training sample. The second label may also be determined manually. For example, the second labels include the classification result determined by a physician, etc.

The preset processing algorithm refers to a preset algorithm for processing the original EMG signals, which is used to obtain the feature information. In some embodiments, the preset processing algorithm may include: performing filter processing and Fourier transform processing on the original EMG signals to obtain the feature information for quantifying the variation condition of the EMG signal during UAB and/or OAB. More descriptions regarding the preset processing algorithm may be found in the related descriptions above.

In some embodiments, the server module 5 may receive the original EMG signals sent by the display interaction module 3, extract the feature information of the original EMG signals, and form a training sample set with the original EMG signals and the feature information to train the intelligent fitting model and the intelligent classification model respectively. The server module 5 may also send the constructed intelligent fitting model and intelligent classification model to the display interaction module 3.

In some embodiments, both the intelligent classification model and the intelligent fitting model are trained based on the machine learning algorithm using the clinical acquisition data as the training sample set. The machine learning algorithm model may use a neural network model, or other models such as a Bayesian polynomial fitting model, which will not be limited here.

In some embodiments, the server module 5 is further configured to: perform filter processing on an original EMG signal of a normal person, an original EMG signal of a patient with OAB symptoms, an original EMG signal of a patient with UAB symptoms, and an original EMG signal of a patient with detrusor hyperactivity with impaired contractility (DHIC) symptoms during a model training phase; and obtain feature information corresponding to original EMG signals during voluntary contraction and involuntary contraction phases based on the preset processing algorithm as an input of the machine learning algorithm.

The normal person refers to a person with normal detrusor activity. The voluntary contraction period refers to a time period during which muscles undergo voluntary contraction. The involuntary contraction period refers to a time period during which muscles undergo involuntary contraction (e.g., passive stretching).

In some embodiments, during the process of model training and construction, the original EMG signals may also be divided into the voluntary contraction EMG signal and the involuntary contraction EMG signal. The voluntary contraction EMG signal refers to an EMG signal during the voluntary contraction period. The involuntary contraction EMG signal refers to an EMG signal during the involuntary contraction period. The data dimensions of the EMG signals and the feature information during the model training phase are the same as those of the original EMG signals and the processed feature information in the aforementioned intelligent diagnostic module 2, which will not be repeated here. For example, the server module 5 may use a peak value of the EMG signal, an average RMS value, etc., as reference values during the involuntary contraction period. For another example, taking the voluntary contraction period as a reference, if the peak value or RMS value during the involuntary contraction period reaches 50% of that during the voluntary contraction period, it can be considered that the patient has symptoms such as detrusor contraction. Furthermore, the judgment of different symptoms may be made based on the feature information of the voluntary contraction EMG signals and the involuntary contraction EMG signals. Of course, the above description regarding the voluntary contraction period and the involuntary contraction period is only an exemplary description. Specific values such as reference values and thresholds need to be set and adjusted according to the actual situation of the patient, which are not limited here.

In some embodiments, the server module 5 is further configured to: designate a diagnostic conclusion and an optimal treatment protocol determined by physicians for each training dataset during the model training phase as an output of the machine learning algorithm, wherein the diagnostic conclusion is used to train the intelligent classification model, and the optimal treatment protocol is used to train the intelligent fitting model.

The diagnostic conclusion refers to the patient's symptoms determined by the physician, including asymptomatic, OAB symptoms, and/or UAB symptoms, etc.

The optimal treatment protocol refers to a preferred treatment protocol evaluated and determined by physicians, including relevant contents such as electrode selection, parameter values of the electrical stimulation therapy, etc.

In some embodiments, the inputs of the intelligent classification model and the intelligent fitting model are the same, i.e., both use the clinical acquisition data of patients as the training sample set. For example, the training sample set includes information such as the amplitude variation of EMG signals, EMG intensity, EMG frequency, and discharge time, etc. However, the labels used for training the intelligent classification model and the intelligent fitting model are different. The label of the intelligent classification model is the classification result of diseases. The purpose of obtaining the output of the intelligent classification model is to classify different symptoms of the patients, while the label of the intelligent fitting model is a treatment protocol with treatment parameters. The server module 5 needs to take the diagnostic conclusion given by the physicians based on the situation of each training sample set as the labels for training the intelligent classification model, and take the optimal treatment protocol given by the physicians based on the situation of each training sample set as the labels for training the intelligent fitting model. For example, in response to determining that there is OAB, the second patch electrode attached to the perianal region may be used for the electrical stimulation therapy. In response to determining that there is UAB, the electrode inserted into the bladder lumen may be used for the electrical stimulation therapy. In response to determining that there is DHIC (both OAB symptom and UAB symptom), dynamic switching from the first treatment mode to the second treatment mode may be realized according to the variation condition in the detrusor activity of the patient, and then the electrode inserted into the bladder lumen or the second patch electrode attached to the perianal region may be used for the electrical stimulation therapy, which is not limited here. Of course, the data dimensions of the diagnostic conclusion or the optimal treatment protocol output during the model training phase are the same as those of the classification result output by the intelligent diagnostic module 2, the first treatment protocol, and the second treatment protocol input by the physicians, which will not be repeated here.

In some embodiments, the intelligent diagnostic module 2 includes an intelligent classification model unit 21 and an intelligent fitting model unit 22. The display interaction module 3 includes an algorithm configuration unit. The algorithm configuration unit 34 is configured to: select and adjust model parameters of the intelligent classification model and the intelligent fitting model; send the intelligent classification model to the intelligent classification model unit 21, and send the intelligent fitting model to the intelligent fitting model unit 22 to perform a model configuration of the intelligent diagnostic module 2.

In some embodiments, the algorithm configuration unit may determine the model parameters for using the intelligent classification model and/or the intelligent fitting model based on the trained parameters and a selection of the user on the terminal device.

The model parameters refer to internal configuration variables of the intelligent classification model and the intelligent fitting model. In some embodiments, the algorithm configuration unit may adjust the model parameters based on the feature information of the original EMG signal and the variation condition of the EMG signal, the medical history data, experiences of the physicians, or the like. For example, if the intensity of the EMG signal of the patient is higher than a threshold for a short period of time, a sensitivity parameter of the intelligent classification model may be reduced to avoid misdiagnosis as OAB. For another example, if the patient had a poor treatment effect of a stimulation parameter in the past treatment, the regression coefficient of the intelligent fitting model may be adjusted.

The intelligent classification model unit 21 is configured to automatically classify symptoms of abnormal bladder activity. In some embodiments, the intelligent classification model unit may automatically output the classification results of abnormal bladder activity (e.g., asymptomatic, OAB, UAB, DHIC, etc.) based on the feature information for quantifying the variation condition of the EMG signal during UAB and/or OAB.

In some embodiments, the display interaction module 3 further includes an original signal unit 31, an EMG signal unit 32, a feature information unit 33, or the like. The original signal unit 31 is configured to display a signal graph of the original EMG signal acquired by the EMG acquisition module 1. The EMG signal unit 32 is configured to display and mark the time domain and frequency domain graphs of the original EMG signals after filter processing. The feature information unit 33 is configured to display the feature information extracted from the original EMG signal. The feature information includes, but is not limited to, the ramp-up phase, maximum value, and ramp-down phase of the EMG signals, the average value during the resting period, the average value of the abdominal surface EMG signals, and the rates of change of these values during the voluntary contraction period and the involuntary contraction period. The algorithm configuration unit 34 is configured to send the intelligent fitting model and/or the intelligent classification model to the intelligent diagnostic module 2. In other words, the user is able to select, via the algorithm configuration unit 34, which model to use in particular, and adjust the model-related parameters according to the actual situation displayed by the display interaction module 3, so that the intelligent diagnostic module 2 may obtain the intelligent fitting model and/or the intelligent classification model configured by the display interaction module 3.

In some embodiments, the display interaction module 3 further includes a treatment protocol configuration unit 35. The treatment protocol configuration unit 35 is configured to: receive the first treatment protocol sent by the intelligent fitting model unit 22, and send the first treatment protocol to the electrical stimulation module 4 to apply the corresponding electrical stimulation pulses. The treatment protocol configuration unit 35 is configured to: receive and display the classification result sent by the intelligent classification model unit 21, and send the second treatment protocol input by the physicians to the electrical stimulation module 4 to apply the corresponding electrical stimulation pulses.

In some embodiments, the treatment protocol configuration unit 35 may receive the first treatment protocol transmitted by the intelligent fitting model unit 22. The physicians may also fine-tune the first treatment protocol through the treatment protocol configuration unit 35, and then transmit the fine-tuned first treatment protocol to the electrical stimulation module 4 for treatment. For example, the physician may fine-tune the first treatment protocol based on the intensity and variation condition of the EMG signals displayed by the treatment protocol configuration unit 35, and the differences in the feature information.

The differences in the feature information refer to varying disparities among the feature information monitored during different periods (i.e., the voluntary contraction period and the involuntary contraction period) for the various types of information included in the feature information.

In some embodiments, the treatment protocol configuration unit 35 may also receive the second treatment protocol input by the physician. Specifically, the treatment protocol configuration unit 35 is configured to display the classification results sent by the intelligent diagnostic module 2. Subsequently, the physician makes a comprehensive judgment based on information such as the original EMG signals, the feature information of the variation condition of the EMG signals, and the classification results, and provides the second treatment protocol. The second treatment protocol is input into the treatment protocol configuration unit 35, and output to the electrical stimulation module 4 by the treatment protocol configuration unit 35.

In some embodiments, the basic parameters of the first treatment protocol and the second treatment protocol include at least one of stimulation waveform, amplitude, pulse width, frequency, ramp-up phase, ramp-down phase, pulse duration, treatment duration, frequency modulation/fixed frequency, high-frequency (HF) range, low-frequency (LF) range, and voltage/current source.

The stimulation waveform, amplitude, pulse width, and frequency refer to a waveform, amplitude, width, and frequency of an electrical stimulation pulse.

The frequency of electrical stimulation pulses includes the HF range and the LF range. In some embodiments, frequencies greater than a threshold (e.g., 50 Hz, etc.) may be determined as the HF range; frequencies less than or equal to the threshold (e.g., 50 Hz, etc.) may be determined as the LF range.

The ramp-up phase refers to time required for the electrical stimulation pulse to rise from a low level (e.g., 10% of the amplitude, etc.) to a high level (e.g., 90% of the amplitude, etc.). The ramp-down phase refers to the time required for the electrical stimulation pulse to drop from a high level (e.g., 90% of the amplitude, etc.) to a low level (e.g., 10% of the amplitude, etc.). The pulse duration refers to duration during which the electrical stimulation pulse remains in the high-level state. In some embodiments, a time period during which the amplitude exceeds 50% of the peak level may be determined as the pulse duration.

The treatment duration refers to a length of time from the start of the electrical stimulation pulse to the end of the electrical stimulation pulse.

The frequency modulation refers to a dynamic change of the frequency of the electrical stimulation pulses over time. In some embodiments, the electrical stimulation pulses with the frequency modulation may reduce tissue adaptability and delay pain tolerance. The fixed frequency refers to a continuous output of electrical stimulation pulses at a fixed frequency, with a constant time interval between adjacent pulses. In some embodiments, the waveform of the electrical stimulation pulses with the fixed frequency is regular and easy to control. In some embodiments, the intelligent diagnostic module may select the frequency modulation or the fixed frequency according to the specific conditions of the patient. For example, if the patient is relatively sensitive to pain, the intelligent diagnostic module may select electrical stimulation pulses with the frequency modulation for treatment.

The voltage/current source of electrical stimulation pulses refers to two output modes of electrical stimulation pulses, which are a voltage source mode and a current source mode. In the voltage source mode, the electrical stimulation module outputs a fixed voltage. In the current source mode, the electrical stimulation module outputs a fixed current.

In some embodiments, the basic parameters of the first treatment protocol and the second treatment protocol may be preset or adjusted according to actual needs.

In some embodiments, the user may adjust the basic parameters in a targeted manner and select an appropriate treatment protocol for treatment, which enhances the capability of precise and personalized treatment. Regardless of which treatment mode is used, the treatment is intelligent and reduces the burden on healthcare professionals.

In some embodiments, the electrical stimulation module 4 is configured to: receive the first treatment protocol and/or the second treatment protocol sent by the display interaction module 3, and apply the corresponding electrical stimulation pulses to the patient according to the first treatment protocol and/or the second treatment protocol.

In some embodiments, in the first treatment mode, the first electrical stimulation unit 41 is configured to: in response to determining that there is UAB, apply the corresponding electrical stimulation pulses by using the electrode inserted into the bladder lumen based on the received first treatment protocol or second treatment protocol. In the second treatment mode, the second electrical stimulation unit 42 is configured to: in response to determining that there is OAB, apply the corresponding electrical stimulation pulses by using the second patch electrode attached to the perianal region based on the received first treatment protocol or second treatment protocol. In response to determining that there is DHIC, the intelligent diagnostic module may automatically realize the dynamic switching from the first treatment mode to the second treatment mode according to the variation condition of the detrusor activity of the patient, and then instruct the electrical stimulation module 4 to perform the electrical stimulation therapy using the electrode inserted into the bladder lumen or the second patch electrode attached to the perianal region, which is not limited here.

It should be noted that the EMG acquisition module 1 and the electrical stimulation module 4 adopt a universal interface. The universal interface may be connected to adhesive electrodes for the vagina, rectum, body surface, and perianal region, and intravesical electrodes, which allows the physician to arbitrarily combine and match the EMG acquisition module 1 and the electrical stimulation module 4 according to actual needs, which will not be repeated here. Certainly, the above descriptions regarding the connection, combination, quantity, and stimulation positions of the electrodes are provided for illustrative purposes only, and not intended to limit the scope of the present disclosure.

FIG. 5 is an exemplary flowchart illustrating generating a first treatment protocol according to some embodiments of the present disclosure. A process 500 may be executed by the processor.

In 510, predicted disease onset data may be determined based on an original EMG signal, feature information, a physiological signal, medical history data, and a symptom manifestation record.

More descriptions regarding the original EMG signal and the feature information may be found in FIG. 1 and relevant descriptions thereof.

The physiological signal refers to a signal related to physiological features of the patient. In some embodiments, the physiological signal may include heart rate, respiratory rate, or the like. The EMG acquisition module may acquire the physiological signal via a smart wearable device, etc. The smart wearable device may include a smart bracelet, or the like.

The medical history data refers to data related to a medical history of the patient. In some embodiments, the medical history data may include past medical history, medication records, or the like. The intelligent diagnostic module 2 may obtain the medical history data based on the medical record of the patient.

The symptom manifestation records refer to records of disease onset in the medical history data of the patient. In some embodiments, the symptom manifestation records may include data such as the frequency, sequence, and time points of the occurrence of various symptoms (e.g., UAB symptoms, OAB symptoms, or DHIC symptoms, etc.). The intelligent diagnostic module 2 may obtain the symptom manifestation record based on the medical record of the patient.

The predicted disease onset data refers to predicted data of a patient's disease onset within a preset future time period. The preset future time period refers to a period of time after the current time. The preset future time period may be manually predefined, such as the next 10 minutes, etc.

In some embodiments, the predicted disease onset data may include the possible symptoms and confidence levels at each time point within the preset future time period, and the estimated feature information at each time point, etc. The confidence level refers to a possibility that a patient will develop symptoms at a specific time point, which may be expressed as a numerical value within a range of 0 to 1. The time point may be predefined, such as each second within the preset future time period. The estimated feature information refers to estimated feature information at each time point within the preset future time period.

In some embodiments, the intelligent diagnostic module 2 may determine the predicted disease onset data through a plurality of manners based on the original EMG signals, feature information, physiological signals, medical history data, and symptom manifestation records. For example, the intelligent diagnostic module 2 may determine the predicted disease onset data through a prediction model, including: preprocessing the original EMG signals, feature information, physiological signals, medical history data, and symptom manifestation records; constructing the preprocessed original EMG signals, feature information, physiological signals, medical history data, and symptom manifestation records into a first feature vector, and inputting the first feature vector into the prediction model to determine the predicted disease onset data.

In some embodiments, the preprocessing may include: performing standardization processing on discrete data such as the original EMG signals, feature information, physiological signals, and symptom manifestation records. For example, the processor records maximum and minimum values of the heart rate, and performs data normalization using the maximum and minimum values as the base (i.e., after normalization, the minimum value=0 and the maximum value=1). The preprocessing may also include: representing categorical features such as the medical history data in the form of one-hot encoding. For example, the processor represents UAB symptoms as 001, OAB symptoms as 010, DHIC symptoms as 100, etc.

In some embodiments, the intelligent diagnostic module 2 constructs the preprocessed original EMG signals, feature information, physiological signals, medical history data, and symptom manifestation records into a first feature vector. For example, the first feature vector may be in the form of [original EMG signals, feature information, physiological signals, medical history data, symptom manifestation records].

In some embodiments, the prediction model may be a machine learning model, such as at least one or a combination of Convolutional Neural Networks (CNN), Recurrent Neural Networks (RNN), etc. An input of the prediction model may include the first feature vector. An output may include the predicted disease onset data.

In some embodiments, the prediction model may be obtained through a large number of third training samples and third labels corresponding to the third training samples. The prediction model is trained in a manner similar to that of the intelligent fitting model, which will not be repeated here.

Each set of training samples in the third training sample may include a historical third vector. The historical third vector is a vector composed of the historical original EMG signals, historical feature information, historical physiological signals, historical medical history data, and historical symptom manifestation records. The third label is the predicted disease onset data corresponding to the third training sample. In some embodiments, the third training sample may be determined from historical data. The third label may be obtained through manual annotation or other approaches. The manual annotation may involve using a disease type and onset time point of historical patients as the third label.

In 520, based on the predicted disease onset data, a predicted symptom feature and a pre-treatment protocol corresponding to the predicted symptom feature may be determined.

The predicted symptom feature refers to a predicted symptom feature of the patient within the preset future time period. In some embodiments, the predicted symptom feature may include the predicted symptom and the predicted treatment time. The intelligent diagnostic module 2 may take the symptom with a confidence level greater than a confidence threshold in the predicted disease onset data as the predicted symptom. The confidence threshold is a corresponding confidence level when a disease occurs. The confidence threshold may be determined in a plurality of ways. For example, the confidence threshold is determined by manual presetting. In some embodiments, the confidence thresholds for different diseases are different, and the confidence thresholds for the same disease at different time points are also different. The predicted treatment time refers to a predicted time point when the disease occurs.

In some embodiments, the confidence threshold is related to the severity of different types of diseases and/or the occurrence time points of diseases in the predicted disease onset data. For example, the confidence threshold is negatively correlated with the severity of different types of diseases. For another example, since a nervous system of the patient is more active during the day, the confidence threshold corresponding to the occurrence time point of the disease being in the daytime (e.g., between 8:00 a.m. and 6:00 p.m.) is higher than that corresponding to the occurrence time point being at night (e.g., between 6:00 p.m. and 8:00 a. m.). The severity of a disease can be determined based on the occurrence frequency of the disease in the disease manifestation records. For example, the higher the frequency, the greater the severity of the disease.

By associating the confidence threshold with the severity of the disease, the system can automatically adjust the stringency of predicting the disease according to the severity of the disease, so as to avoid missed diagnosis or misdiagnosis. By dynamically setting the confidence threshold in combination with the occurrence time point of the disease, the treatment protocol can be accurately determined for the risk of disease onset in different time periods.

The pre-treatment protocol refers to a treatment protocol determined based on the predicted symptom feature. In some embodiments, the pre-treatment protocol may include at least one of a first pre-treatment protocol and a second pre-treatment protocol, etc. The intelligent diagnostic module 2 may input the feature information corresponding to the predicted disease in the predicted symptom feature into the intelligent fitting model to obtain the first pre-treatment protocol. The intelligent diagnostic module 2 may also send the estimated feature information to the physician through the display interaction module and receive the second pre-treatment protocol set by the physician. More descriptions regarding the intelligent fitting model may be found in FIG. 1 and relevant descriptions thereof.

In 530, in response to the actual symptom feature and the predicted symptom feature meeting a pre-treatment condition, the first treatment protocol may be generated based on the pre-treatment protocol.

The actual symptom feature refers to a disease feature that actually appears in the patient within a preset time period. The preset time period refers to a preset future time period determined when formulating the pre-treatment protocol. In some embodiments, the actual symptom feature may include the actual disease that occurs in the patient within the preset time period and the time point when the actual disease occurs. The intelligent diagnostic module 2 may obtain the actual symptom feature in real time based on physician feedback, or the like. By invoking the intelligent fitting model in advance to prepare the treatment protocol and real-time monitoring of the patient's status within the preset time period corresponding to the treatment protocol, the treatment protocol can be implemented quickly when the patient's disease occurs.

The pre-treatment condition refers to a condition under which the pre-treatment protocol is used as the first treatment protocol. In some embodiments, the pre-treatment condition may be that the actual disease is consistent with the predicted disease, and the difference between the time point when the actual disease occurs and the time point corresponding to the predicted disease is within a preset time threshold. The preset time threshold may be determined by manual presetting. For example, the preset time threshold is 10 seconds, etc.

In some embodiments, in response to the actual symptom feature and predicted symptom feature meeting the pre-treatment condition, the intelligent diagnostic module 2 may take the pre-treatment protocol as the first treatment protocol.

In some embodiments of the present disclosure, the accuracy of disease prediction is improved by fusing multi-source data and using prediction models to predict diseases and their onset times in advance. By invoking the intelligent fitting model in advance to prepare the treatment protocol, the pre-treatment protocol can be directly applied at the moment symptoms are detected, which prevents delayed treatment caused by determining the treatment protocol only when diseases occur, thereby enhancing the timeliness of treatment. Additionally, planning the treatment protocol in advance reduces the risk of acute disease episodes and improves the initiative in treatment.

FIG. 6 is an exemplary schematic diagram illustrating a feature extraction model according to some embodiments of the present disclosure.

In some embodiments, the intelligent diagnostic module is further configured to: obtain feature information 630 through a feature extraction model 620 based on an original EMG signal 611, medical history data 612, a patient's voiding log 613, and patient information 614, etc.

The patient's voiding log refers to data that records the voiding history of the patient. The patient's voiding log may include a count of times of urination within a second preset time period (e.g., one day, two days, etc.), a volume of each urination, etc.

In some embodiments, the intelligent diagnostic module may obtain the patient's voiding log based on the medical record, input from an operator, or the like.

The patient information may include age, weight, gender, etc., of the patient. In some embodiments, the intelligent diagnostic module may obtain the patient information based on the medical record, input from an operator, or the like.

More descriptions regarding the original EMG signal may be found in FIG. 1 and relevant descriptions thereof. More descriptions regarding the medical history data may be found in FIG. 5 and relevant descriptions thereof.

The feature extraction model refers to a model used to extract the feature information.

In some embodiments, the feature extraction model may be a machine learning model. For example, the feature extraction model includes any one or a combination of a Convolutional Neural Network (CNN) model, a Temporal Convolutional Network (TCN) model, etc., or other custom model structures.

In some embodiments, the processor may preprocess the original EMG signals, the medical history data, the patient's voiding logs, and the patient information. The processor constructs a second feature vector based on the preprocessed data, and inputs the second feature vector into the feature extraction model 620 to extract the feature information 630.

The process of the preprocessing is similar to that of the preprocessing involved in FIG. 5. More content may be found in FIG. 5 and relevant descriptions thereof.

In some embodiments, the intelligent diagnostic module may construct the preprocessed original EMG signals, the medical history data, the patient's voiding log, and the patient information as the second feature vector.

In some embodiments, the feature extraction model 620 may be trained using a large number of fourth training samples with fourth labels. Each set of training samples in the fourth training samples includes a sample second feature vector. The fourth labels may be the sample feature information corresponding to the fourth training sample.

In some embodiments, the intelligent diagnostic module may construct a historical second vector from historical original EMG signals, historical medical history data, historical patient's voiding logs, and historical patient information obtained from historical data, and use the historical feature vectors as the fourth training sample. The intelligent diagnostic module uses the feature information manually labeled in the historical data as the fourth label, or the feature information obtained by performing filter processing and Fourier transform processing on the historical original EMG signals in the historical data as the fourth label.

More descriptions regarding the filter processing and the Fourier transform processing may be found in FIG. 1 and relevant descriptions thereof.

The feature extraction model is trained in a manner similar to that of the intelligent fitting model, which will not be repeated here.

By introducing the feature extraction model and integrating multi-dimensional data such as the patient's voiding log, the patient information, etc., the comprehensiveness and intelligence of the feature information extraction are enhanced to accommodate complex cases. The feature extraction model mines deep data correlations to uncover the feature information that is difficult to identify using traditional manners, which helps improve the accuracy and reliability of diagnosing bladder dysfunction.

In some embodiments, the intelligent interactive neuromodulation system by intravesical and pelvic floor stimulation further includes a pressure monitoring module. The pressure monitoring module is configured to monitor bladder pressure. The input to the feature extraction model further includes the bladder pressure.

In some embodiments, the pressure monitoring module may include a wearable bladder pressure monitoring device, a wireless bladder pressure monitoring device, or the like.

The wearable bladder pressure monitoring device refers to a portable bladder pressure monitoring device that monitors the bladder pressure of the patient in real time by wearing. For example, the wearable bladder pressure monitoring device includes a wearable ultrasonic patch, a wearable scanner system, or the like.

The wireless bladder pressure monitoring device refers to a bladder pressure monitoring device that sends data to an external receiving terminal (e.g., a smartphone, a hospital monitoring system, etc.) via wireless transmission technologies (e.g., Bluetooth, wireless network, or dedicated radio frequency). The wireless bladder pressure monitoring devices may be wearable or implantable, such as a bladder pressure monitor, etc.

The bladder pressure (also referred to as intravesical pressure) refers to internal pressure exerted on the bladder by the urine within the bladder.

In some embodiments, the intelligent diagnostic module may preprocess the bladder pressure, add the preprocessed data to the above second feature vector, and input the second feature vector into the feature extraction model to extract the feature information.

In some embodiments, when the input to the feature extraction model includes the bladder pressure, the historical second feature vector is further constructed based on historical bladder pressure. The constructed historical second feature vector is used as the fourth training sample.

By adding the bladder pressure monitoring and incorporating the monitored bladder pressure into the input of the feature extraction model, joint analysis of the EMG signals and the bladder pressure can be achieved, comprehensively reflecting the physiological state of the bladder and yielding more accurate feature information.

In summary, the beneficial effects that may be brought about by the embodiments of the present disclosure include, but are not limited to:

The intelligent interactive neuromodulation system by intravesical and pelvic floor stimulation of the embodiments of the present disclosure can independently evaluate the current clinical feature information of the patient by collecting, processing, and identifying the original EMG signals of the patient, so as to realize the adaptive dynamic switching between the treatment modes for UAB symptoms and OAB symptoms, and timely adopt corresponding treatment protocols. The system eliminates the need for the physicians to monitor the status of the patient in real time, which greatly reduces the burden on the physicians.

The intelligent interactive neuromodulation system by intravesical and pelvic floor stimulation of the embodiments of the present disclosure uses the intelligent fitting model to directly output the first treatment protocol, which is transmitted to the electrical stimulation module through the display interaction module. By automatically generating the first treatment protocol for electrical stimulation, there is no need for frequent manual adjustments, which effectively improves the treatment efficiency and effect.

The intelligent interactive neuromodulation system by intravesical and pelvic floor stimulation of the embodiments of the present disclosure obtains corresponding classification results according to the intelligent classification model and the feature information representing detrusor activity. The physicians comprehensively evaluate and provide the second treatment protocol based on information such as the original EMG signals, feature information and classification results displayed by the display interaction module, so as to perform the electrical stimulation therapy more effectively and accurately.

The intelligent interactive neuromodulation system by intravesical and pelvic floor stimulation of the present disclosure adopts two manners to obtain two treatment protocols (i.e., the first treatment protocol obtained through the intelligent fitting model and the second treatment protocol input by the physicians based on the classification results of the intelligent classification model). The system can selectively choose an appropriate treatment protocol according to the dynamic switching between treatment modes, and has better capabilities in precise and personalized treatment. No matter which treatment manner is adopted, the system achieves intelligent treatment, reducing the burden on healthcare professionals.

It should be noted that in the present disclosure, unless otherwise explicitly defined or limited, terms such as “connection” and “fixation” should be understood in a broad sense. For example, it can be a fixed connection, a detachable connection, or an integral connection; it can be a mechanical connection, an electrical connection, or mutual communication. It can be a direct connection, or an indirect connection through an intermediate medium. It can be the internal communication between two components or the interaction relationship between two components, unless otherwise clearly defined. For those skilled in the art, the specific meanings of the above terms in the present disclosure can be understood according to specific situations.

It should also be noted that the terms “comprise”, “include” or any other variants thereof are intended to cover non-exclusive inclusion, so that a process, method, commodity or device including a series of elements not only includes those elements, but also includes other elements not explicitly listed, or elements inherent in such a process, method, commodity or device. Without more restrictions, an element defined by the sentence “including a . . . ” does not exclude the existence of other identical elements in the process, method, commodity or device including the element.

It should be noted that the above descriptions are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

What is claimed is:

1. An intelligent interactive neuromodulation system by intravesical and pelvic floor stimulation, wherein the system comprises: an electromyography (EMG) acquisition module, an intelligent diagnostic module, a display interaction module, and an electrical stimulation module, the electrical stimulation module includes a first electrical stimulation unit and a second electrical stimulation unit, the EMG acquisition module is connected with the intelligent diagnostic module and the display interaction module, the electrical stimulation module is connected with the display interaction module, wherein the EMG acquisition module includes a first patch electrode attached to a perianal region, the first electrical stimulation unit includes an electrode inserted into the bladder lumen, the second electrical stimulation unit includes a second patch electrode attached to the perianal region;
the intelligent diagnostic module is configured to:
receive an original EMG signal acquired by the EMG acquisition module;
obtain, based on the original EMG signal, feature information for quantifying a variation condition of the EMG signal during underactive bladder (UAB) and/or overactive bladder (OAB);
generate a first treatment protocol by using the feature information and a preconfigured intelligent fitting model, and generate a classification result of an abnormal bladder activity based on the feature information and a preconfigured intelligent classification model;
in response to identifying that a detrusor activity of a patient changes, automatically complete dynamic switching between a first treatment mode and a second treatment mode, wherein
the first treatment mode includes: in response to determining that there is UAB, applying corresponding electrical stimulation pulses by using the electrode inserted into the bladder lumen based on the received first treatment protocol or a second treatment protocol input by physicians;
the second treatment mode includes: in response to determining that there is OAB, applying corresponding electrical stimulation pulses by using the second patch electrode attached to the perianal region based on the received first treatment protocol or the second treatment protocol input by the physicians;
the intelligent diagnostic module is further configured to:
in response to determining that the patient presents with symptoms of UAB, use the first treatment mode to perform electrical stimulation therapy using the electrode inserted into the bladder lumen;
in response to determining that an EMG activity of the patient changes, determine that a current UAB is changed to the OAB, automatically switch and use the second treatment mode, deactivate the electrical stimulation pulses by using the electrode inserted into the bladder lumen, and activate the electrical stimulation pulses by using the second patch electrode attached to the perianal region; and
in response to determining that the EMG activity of the patient changes, determine that a current OAB is changed to the UAB, automatically switch and use the first treatment mode, close the second patch electrode attached to the perianal region, and activate the electrical stimulation pulses by using the electrode inserted into the bladder lumen.

2. The intelligent interactive neuromodulation system of claim 1, wherein the display interaction module is configured to:
receive the first treatment protocol, send the first treatment protocol to the electrical stimulation module to apply the corresponding electrical stimulation pulses;
or, the display interaction module is further configured to:
receive the second treatment protocol input by the physicians based on the original EMG signal, the feature information, and the classification result received by the display interaction module, and send the second treatment protocol to the electrical stimulation module to apply the corresponding electrical stimulation pulses.

3. The intelligent interactive neuromodulation system of claim 1, wherein the system further includes a server module, the server module is connected with the display interaction module, the server module is configured to:
construct the intelligent fitting model and/or the intelligent classification model, and send a trained intelligent fitting model and/or intelligent classification model to the display interaction module;
wherein the intelligent fitting model and/or the intelligent classification model is trained by a training sample set via a machine learning algorithm, training data in the training sample set includes clinical acquisition data, including an original EMG signal sent by the display interaction module and feature information of the original EMG signal processed by a preset processing algorithm.

4. The intelligent interactive neuromodulation system of claim 3, wherein the server module is further configured to:
perform a filter processing on an original EMG signal of a normal person, an original EMG signal of a patient with OAB symptoms, an original EMG signal of a patient with UAB symptoms, and an original EMG signal of a patient with detrusor hyperactivity with impaired contractility (DHIC) symptoms during a model training phase; and
obtain feature information corresponding to original EMG signals during voluntary contraction and involuntary contraction phases based on the preset processing algorithm as an input of the machine learning algorithm.

5. The intelligent interactive neuromodulation system of claim 4, wherein the server module is further configured to:
designate a diagnostic conclusion and an optimal treatment protocol determined by physicians for each training dataset during the model training phase as an output of the machine learning algorithm, wherein the diagnostic conclusion is used to train the intelligent classification model, and the optimal treatment protocol is used to train the intelligent fitting model.

6. The intelligent interactive neuromodulation system of claim 1, wherein the intelligent diagnostic module includes an intelligent classification model unit and an intelligent fitting model unit, and the display interaction module includes an algorithm configuration unit,
the algorithm configuration unit is configured to:
select and adjust model parameters of the intelligent classification model and the intelligent fitting model; and
send the intelligent classification model to the intelligent classification model unit, and send the intelligent fitting model to the intelligent fitting model unit to perform a model configuration of the intelligent diagnostic module.

7. The intelligent interactive neuromodulation system of claim 6, wherein the display interaction module further includes a treatment protocol configuration unit, and the treatment protocol configuration unit is configured to:
receive the first treatment protocol sent by the intelligent fitting model unit, and send the first treatment protocol to the electrical stimulation module to apply the corresponding electrical stimulation pulses;
or, the treatment protocol configuration unit is configured to:
receive and display the classification result sent by the intelligent classification model unit, send the second treatment protocol input by the physicians to the electrical stimulation module to apply the corresponding electrical stimulation pulses.

8. The intelligent interactive neuromodulation system of claim 1, wherein the intelligent diagnostic module is further configured to:
perform filter processing and Fourier transform processing on the original EMG signal to obtain the feature information for quantifying the variation condition of the EMG signal during UAB and/or OAB;
wherein the filter processing is used to extract an EMG signal in a frequency range of 20 Hz-500 Hz from the original EMG signal, and signal instantaneous root mean square (RMS) values are calculated according to a preset sampling rate to perform window smoothing processing, an RMS variation curve after window smoothing processing is used to extract the feature information.

9. The intelligent interactive neuromodulation system of claim 1, wherein the original EMG signal includes at least one of a vaginal surface EMG signal, an anal sphincter and perianal region EMG signal, and an abdominal surface EMG signal of a patient.

10. The intelligent interactive neuromodulation system of claim 1, wherein basic parameters of the first treatment protocol and the second treatment protocol include at least one of stimulation waveform, amplitude, pulse width, frequency, ramp-up phase, ramp-down phase, pulse duration, treatment duration, frequency modulation/fixed frequency, high-frequency (HF) range, low-frequency (LF) range, and voltage/current source.
